(19) United States
(12) Patent Application Publication
(10) Pub. No.: US 2022/0333940 A1
(43) Pub. Date: Oct. 20, 2022

(54) MONITORING AN AMBIENT AIR PARAMETER USING A TRAINED MODEL

(71) Applicant: Ecosystem Informatics Inc., Mississauga (CA)

(72) Inventors: Shirook ALI, Milton (CA); Mohamed BAKR, Oakville (CA); Houssam KANJ, Waterloo (CA)

(21) Appl. No.: 17/718,804

(22) Filed: Apr. 12, 2022

Related U.S. Application Data
(60) Provisional application No. 63/174,880, filed on Apr. 14, 2021.

(30) Foreign Application Priority Data
Aug. 5, 2021 (CA) PCT/CA2021/051086

(51) Int. Cl. G01C 21/34; B60W 40/105

(52) U.S. Cl. CPC G01C 21/3469; G01C 21/3461; B60W 40/105; G06K 9/6256

(57) ABSTRACT

A method and related system are provided for estimating or predicting an output value of a first ambient parameter (AP). Sensors generate a plurality of time-paired training values of the first AP, a second AP, and accuracy factor(s) affecting the accuracy of the measured first or second APs. A computer uses the training values, and trust coefficients based on the accuracy factor to train an air quality prediction model for estimating or predicting the output value of the first AP based on a measured input value of the second AP. The computer estimates or predicts the output value of the first AP by applying the trained air quality prediction model to the input value of the second AP. The estimated or predicted output value of the first AP may be used to calibrate the accuracy of measured values of the first AP, or forecast future values of the first AP.

### CROSS-REFERENCE TO RELATED APPLICATIONS

The present applicant claims the priority benefit of U.S. Provisional Patent Application 63/174,880, filed on Apr. 14, 2021, and PCT International Application PCT/CA2021/051086, filed on Aug. 5, 2021, the entire contents of both which are incorporated herein by reference.

### FIELD OF THE INVENTION

The present invention relates to monitoring of ambient air parameters.

### BACKGROUND OF THE INVENTION

Ambient air parameters, such as the concentration of gaseous air pollutants and air temperature, may be monitored for a variety of reasons, such as alerting populations of health risks, evaluating compliance with air quality standards, and mapping air quality patterns.

Sensors may be stationary or mobile, however, a sensor mounted on a moving vehicle could monitor air quality over a greater geographic area than would be possible if the sensor were stationary. However, changing vehicle motion results in temperature, wind direction, and wind speed variations, which can affect the accuracy of the sensor readings.

Different sensors may be used to monitor different ambient parameters. However, increasing the number of different sensors increases the complexity of a monitoring system, and the amount of the sensor data that needs to be transmitted. This has practical implications for cost, efficiency, and reliability when monitoring is performed on a large scale.

There remains a need in the art for accurate, economical, and efficient monitoring of multiple ambient parameters, particularly using sensors mounted on mobile platforms.

### SUMMARY OF THE INVENTION

In one aspect, the invention may comprise a method for estimating or predicting an output value of a first ambient parameter (AP), such as NO₂ concentration, O₃ concentration, or air temperature, based on at least one measured input value of a second AP, wherein the second AP is different from the first AP, the method comprising the steps of:
(a) generating a plurality of time-separated training values of the first AP, a plurality of time-separated training values of the second AP, and a plurality of time-separated values of an accuracy factor that affects an accuracy of measured values of either one or both of the first and second APs, wherein each of the training values of the second AP and the accuracy factor are time-paired uniquely with one of the training values of the first AP;
(b) using a computer system, for each of the time-paired training values of the first and second APs, calculating an associated trust coefficient based on the value of the accuracy factor that is uniquely time-paired to the training values of the first and second APs;
(c) using the computer system, training an air quality estimation/prediction model for estimating or predicting the output value of the first AP based on the at least one measured input value of the second AP, wherein the training is based on the training values of the first and second APs weighted by the associated trust coefficients;
(d) measuring the at least one input value of the second AP; and
(e) using the computer system, estimating or predicting the output value of the first AP by applying the trained air quality estimation/prediction model to the at least one measured input value of the second AP.

The trust coefficient may be based on any accuracy factor which affects the accuracy of measured values of either one or both the first and second APs. In one embodiment, the accuracy factor comprises the speed of a mobile platform, which may be determined by a GPS module or a telematics system, used in association with a first sensor for measuring the first AP training values, and a second sensor for measuring the second AP training values. In other embodiment, the accuracy factor may comprise wind speed, air temperature, ambient air pressure, relative humidity, or a combination of any of the foregoing.

In some embodiments, the method is implemented on a mobile platform, for example where the first AP training values are measured by a first sensor, and the second AP training values and at least one measured input value of the second AP are measured by a second sensor, which are both mounted to a vehicle. Alternatively, the second AP training values and at least one measured input value of the second AP may be measured by a second sensor and a third sensor respectively.

In some embodiments, step (e) of the method estimates the output value of the first AP for a time corresponding to when at least one measured input value of the second AP is measured. In such embodiments, the method may comprise the further step of using the estimated output value of the first AP to calibrate the accuracy of a measured test value of the first AP. Alternatively, in other embodiments, step (e) of the method predicts the output value of the first AP for a time subsequent to when at least one measured input value of the second AP is measured. That is, at least one measured input value of the second AP may be a historical measured value of the second AP, and the method may comprise the step of using the predicted output value of the first AP for forecasting purposes.

In some embodiments, the air quality estimation/prediction model comprises a machine learning module, such as a deep learning module, such as an artificial neural network (ANN). The ANN may comprise at least one dense layer. Preferably, the ANN further comprises an input data scaling block, at least one long short-term memory block, and an output data inverse scaling block.

In some embodiments, the method comprises the further step of periodically augmenting the air quality estimation/prediction model with a measured or artificial data set.

In another aspect, the invention may comprise a system for estimating or predicting an output value of a first ambient parameter (AP), such as NO₂ concentration, O₃ concentration, or air temperature, based on at least one measured input value of a second AP, such as CO concentration, that is different from the first AP, the system comprising:
a first sensor for measuring values of the first AP;
a second sensor for measuring values of a second AP;
an accuracy factor sensor for measuring values of at least one accuracy factor that affects an accuracy of measured values of either one or both of the first and second APs;
a computer system comprising: a processor operatively connected to the first sensor to access the values of the first AP, to the second sensor to access values of the second AP, and to the accuracy factor sensor to access values of the accuracy factor; and a non-transitory computer readable medium storing instructions that, when executed by the processor, cause the processor to perform a method comprising the steps of:
(a) accessing a plurality of time-separated training values of the first AP, the second AP, and the accuracy factor, wherein each of the training values of the second AP and the accuracy factor are time-paired uniquely with one of the training values of the first AP;
(b) for each of the time-paired training values of the first and second APs, calculating an associated trust coefficient based on the value of the accuracy factor that is uniquely time-paired with the training values of the first and second APs;
(c) training an air quality estimation/prediction model for estimating or predicting the output value of the first AP based on the at least one measured input value of the second AP, wherein the training is based on the time-paired training values of the first and second APs weighted by the associated trust coefficients;
(d) accessing the at least one measured input value of the second AP; and
(e) estimating or predicting the output value of the first AP by applying the trained air quality estimation/prediction model to the at least one measured input value of the second AP.

The system may be configured to implement any embodiment of a method described herein. In some embodiments, at least the first and second sensors are mounted on a mobile platform, and the system further comprises a GPS module or other vehicle telemetry for determining speed of the mobile platform.

In different embodiments, the present invention may comprise a method or system comprising any combination of elements or features described herein, or which specifically omits any particular feature or element described herein.

### BRIEF DESCRIPTION OF THE DRAWINGS

In the drawings, like elements may be assigned like reference numerals. The drawings are not necessarily to scale, with the emphasis instead placed upon the principles of the present invention. Additionally, each of the embodiments depicted are but one of a number of possible arrangements utilizing the fundamental concepts of the present invention.

FIG. 1 is a schematic diagram of an embodiment of a system of the present invention for monitoring air quality.

FIG. 2 is a schematic block diagram of an embodiment of a sensor unit of the system of FIG. 1.

FIG. 3 is a schematic block diagram of an embodiment of a server computer of the system of FIG. 1.

FIG. 4 is a flow chart of an embodiment of a method of the present invention for monitoring air quality.

FIG. 5 is a chart showing an example of speed variation for a vehicle-mounted sensor unit over a plurality of time-separated air quality samples collected over a vehicle trip.

FIG. 6 is a schematic depiction of a first embodiment of an air quality estimation/prediction model used in the present invention.

FIG. 7 is a schematic depiction of a second embodiment of air quality estimation/prediction model used in the present invention.

FIG. 8 is a schematic depiction of an example of data augmentation of the air quality estimation/prediction model.

### DETAILED DESCRIPTION OF EMBODIMENTS OF THE INVENTION

### Definitions

The invention relates to monitoring air quality and/or atmospheric condition. Any term or expression not expressly defined herein shall have its commonly accepted definition understood by a person skilled in the art. As used herein, the following terms have the following meanings.

“Ambient parameter” or “AP” refers to any physically measurable property of air. In embodiments, the AP may be a concentration of a gaseous component of air with non-limiting examples of such gaseous components being carbon monoxide (CO), carbon dioxide (CO₂), nitrous oxide (NO), nitrogen oxides of the formula NOₓ such as nitrogen dioxide (NO₂), ozone (O₃), methane (CH₄), and sulfur oxides of the form SO_{X} such as sulfur dioxide (SO₂) In other embodiments, the AP may be a concentration of suspended particulate matter in general, or a concentration of suspended particulate matter of a specific composition such as lead. In still other non-limiting embodiments, the AP may be a weather condition, such as air temperature, humidity, barometric pressure, and wind speed.

“Air quality estimation/prediction model” refers to one or more rules (e.g., expressed by mathematical formulae, and/or logical relationships), stored in a memory, that determine an output comprising an estimated or predicted value of a first ambient parameter based on an input comprising a value of a second ambient parameter that is different from the first ambient parameter.

“GPS module” refers to a device that includes an antenna for receiving satellite navigation signals (e.g., signals transmitted by the Global Positioning System (GPS), the Global Navigation Satellite System (GLONASS), the Galileo positioning system, the Beidou Navigation Satellite System, or satellite navigation systems), and an operatively connected processor that is configured with a set of instructions stored on a memory, to analyze such signals to determine the location of the module, and optionally, other kinematic information such as distance travelled, direction of movement, speed, and acceleration of the module. GPS modules are known in the art, and do not, by themselves constitute the present invention. Persons skilled in the art may refer to a satellite navigation signal receiver module as a “GPS receiver,” or a “GNSS receiver,” depending on the type of satellite navigation signal used by the module.

“Memory” refers to a non-transitory tangible computer-readable medium for storing information in a format readable by a processor, and/or instructions readable by a processor to implement an algorithm. The term “memory” includes a plurality of physically discrete, operatively connected devices despite use of the term in the singular. Non-limiting types of memory include solid-state, optical, and magnetic computer readable media. Memory may be non-volatile or volatile. Instructions stored by a memory may be based on a plurality of programming languages known in the art, with non-limiting examples including the C, C++, Python™, MATLAB™, and Java™ programming languages.

“Mobile platform” or “mobile sensor” refers to any device including a sensor which is not in a fixed stationary geolocation. Mobile platforms may include any consumer or industrial means of transportation, such as automobiles, trucks, buses, trains, tractors, motorcycles, or bicycles, whether powered or not. Mobile platforms may further include handheld or portable devices which are manually moved, such as by a pedestrian.

“Processor” refers to one or more electronic devices that is/are capable of reading and executing instructions stored on a memory to perform operations on data, which may be stored on a memory or provided in a data signal. The term “processor” includes a plurality of physically discrete, operatively connected devices despite use of the term in the singular. Non-limiting examples of processors include devices referred to as microprocessors, microcontrollers, central processing units (CPU), and digital signal processors.

“Training”, refers to a process, implemented by a processor according to an algorithm stored in a memory, that determines the value(s) of one or more variable(s) of the rule(s) defining an air quality estimation/prediction model, based on a training dataset of known values of first and second ambient parameters, in order to increase the accuracy of the value of the first ambient parameter estimated or predicted by the air quality estimation/prediction model.

### Overview

The present invention exploits observable correlations between values of at least one first ambient parameter (AP) (e.g., nitrogen dioxide (NO₂) concentration, ozone (O₃) concentration, and/or air temperature (T)), and a different second AP (e.g., carbon monoxide concentration) by using them as “training” values for training an air quality estimation/prediction model, and applying the trained air quality estimation/prediction model to estimate or predict an “output” value for the at least one first AP based on measured “input” value(s) of the second AP.

In one embodiment, the present invention can be used to estimate an output value of the first AP for a time corresponding to when the at least one measured input value of the second AP is measured, in order to calibrate or evaluate the accuracy of a measured test value of the first AP. Thus, in some embodiments, the present invention may be useful when the accuracy of measured values of the first AP is expected to be lower or less stable than the accuracy of measured values of the second AP, under anticipated conditions such as changing temperature, wind speed, wind direction, ambient air pressure, and/or relative humidity conditions, such as those that may be encountered by sensors mounted on a moving mobile platform or forming part of a mobile sensor.

In some embodiments, the present invention can be used to predict an output value of the first AP for a time that is subsequent to when the measured input value(s) of the second AP/are measured. Thus, in some embodiments, the present invention may be useful where it is desired to forecast the first AP, before the first AP is measured.

The present invention allows the value of the first AP to be estimated or predicted based on a measured input value of the second AP, without measuring the first AP beyond the training values. Thus, the present invention may be useful where it is desired to reduce measurements of the first AP, the need for sensor equipment to measure the first AP, and/or the amount of sensor data for the first AP that is to be transmitted from the sensor equipment.

### System

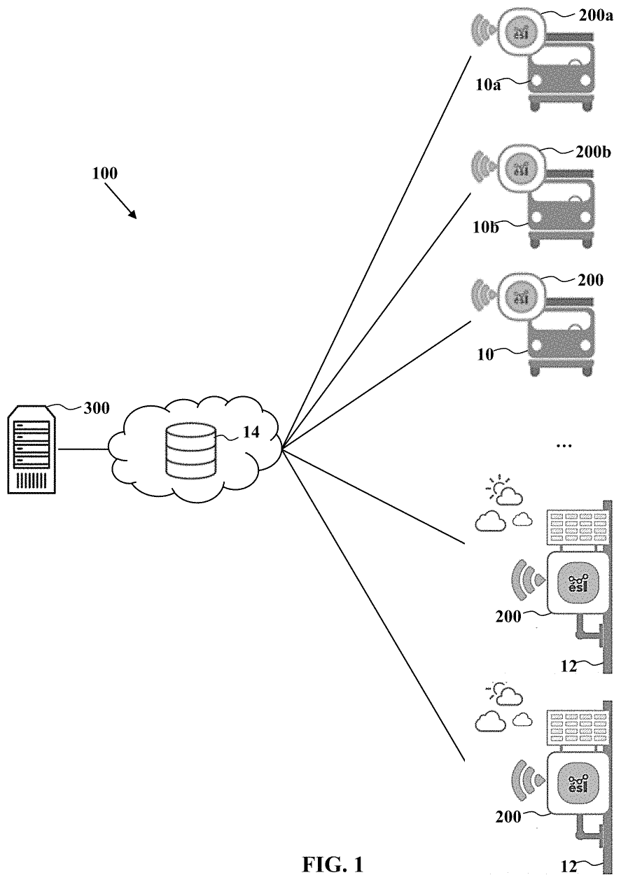

FIG. 1 is a schematic diagram of an embodiment of a system (100) for monitoring pollution levels and air quality as well as weather information, comprising at least one sensor unit (200), and a computer system. In this embodiment, the computer system comprises the server computer (300), which is in a server-client relationship with each sensor unit (200). In this embodiment, some of sensor units (200) are mobile sensors or are mounted on mobile platforms, in this case in the form of vehicles (10), and others are mounted on a stationary structure (12) (e.g., a street lighting pole, or a building), but the present invention is not limited by the number of sensor units (200) or how they are deployed. In this embodiment, server computer (300) is capable of accessing sensor data generated by the sensors of sensor units (200) via a communications network (e.g., the Internet, or an intranet) as represented by the connecting lines in FIG. 1. In one embodiment, sensor units (200) wirelessly transmit sensor data via the communications network to a memory of a cloud database (14), which stores the sensor data so that it can be accessed by server computer (300). In other embodiments, the sensor units (200) may transmit some or all of the sensor data via a communications network to a memory that is local to server computer (300). In still other embodiments, the computer system may be physically connected to sensor unit (200), and the sensor unit (200) may store some or all of the sensor data to a memory that is local to the sensor unit (200), so that it may be processed by a processor that is local to the sensor unit (200), in an “offline mode” without the need for a communications network.

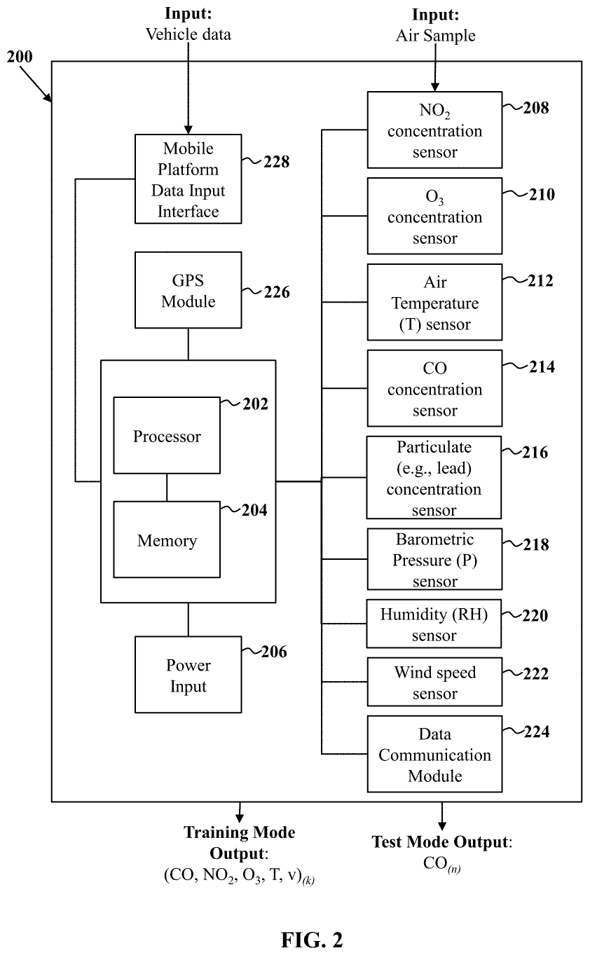

FIG. 2 is a schematic block diagram of an embodiment of a sensor unit (200), comprising a processor (202) operatively connected to a memory (204), a power input (206), and a plurality of sensors, which may include a nitrogen dioxide concentration sensor (208), an ozone concentration sensor (210), an air temperature sensor (212), carbon monoxide concentration sensor (214), particulate concentration (e.g. lead concentration) sensor (216), a barometric pressure sensor (218), a relatively humidity (RH) sensor (220), a wind speed sensor (222), a data communication module (224), a GPS module (226), and a mobile platform data input interface (228). The sensors (208 to 222) are physically proximate to each other, such that they are reading the air quality in substantially the same location. In one embodiment, for example, the sensors (208 to 222) are attached to a common housing that is to be mounted on mobile platform such as a vehicle (10), form part of an integrated mobile sensor, or stationary structure (12).

In one embodiment, processor (202) and memory (204) collectively form a microcontroller, with firmware coded in memory (204) configuring processor (202) to control sensors (208 to 222) for acquisition of sensor data, and to control data communication module (224) for transmission of sensor data via a communications network. In one embodiment, power input (206) may receive electrical power from a supply such as a battery of the sensor unit (200) or a vehicle or other type of mobile platform, or a solar panel. Sensors (208 to 222) may be implemented by digital electrochemical sensors, thermometers, pressure sensors, hygrometers, anemometers, and other sensor devices known in the art that are capable of measuring the applicable AP of interest to generate sensor data in digital form. Data communication module (224) may comprise any combination of hardware and/or software that allows for transmission of the sensor data from sensor unit (200) via a communications network. In one embodiment, data communication module (224) may comprise a cellular modem and antenna for wireless transmission of sensor data to the communications network. GPS module (226) generates data indicative of the location of the sensor unit (200), and optionally other kinematic data of the sensor unit (200), such as its distance travelled, direction of movement, speed, and/or acceleration. Mobile platform data input interface (228) may comprise any combination of hardware and software for receiving data from a system of a mobile platform to which the sensor unit (200) may be mounted. For example, in embodiments where the mobile platform is a vehicle or a portable computer equipped with its own GPS module and/or telematics system for monitoring the mobile platform's location, distance travelled, direction of movement, speed, and/or acceleration, the mobile platform data input interface may comprise a wired or wireless databus for communication with such GPS module and/or telematics system. Thus, the sensor unit (200) may “piggyback” on the mobile platform's GPS module and/or telematics system, and the sensor unit (200) need not have its own GPS module (226).

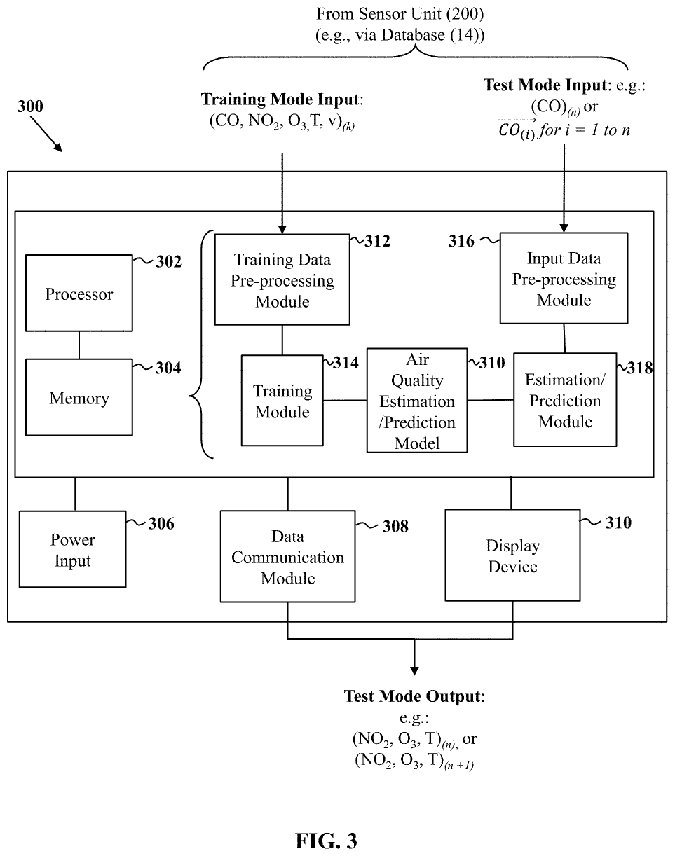

FIG. 3 is a schematic block diagram of an embodiment of a server computer (300) comprising a processor (302) operatively connected to a memory (304), a power input (306), a data communication module (308), and optionally a display device (310). In one embodiment, processor (302) may be implemented by a CPU, and memory (304) may be implemented by a hard drive or solid-state memory. Memory (304) stores an air quality estimation/prediction model (310), and a set of instructions that are conceptualized in FIG. 3 as a training data pre-processing module (312), a training module (314), an input data pre-processing module (316), and an estimation/prediction module (318). These instructions are executable by processor (302) to implement steps of the method of the present invention as described below. Data communication module (308) may comprise any combination of hardware and/or software that allows for reception of sensor data, and transmission of output data via a communications network. In one embodiment, data communication module (308) may comprise an Internet modem. Display device (310) may be any device that allows for display of data in text and/or graphical form. In one embodiment, the display device (310) is a computer display monitor.

### Method

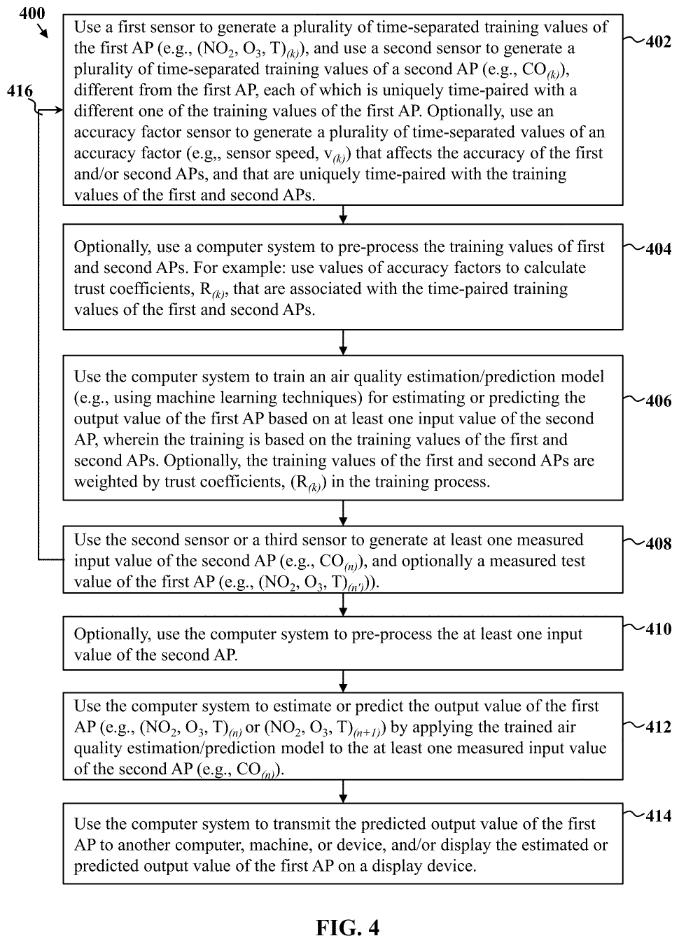

FIG. 4 is a flow chart of an embodiment of a method (400) of the present invention for estimating or predicting output values of first ambient parameter(s) (“first APs”) based on a measured input value of a second ambient parameter (“second AP”). In this embodiment, each of nitrogen dioxide (NO₂) concentration, ozone (O₃) concentration, and air temperature (T) is a “first AP”, while carbon monoxide concentration (CO) is the “second AP”. The method is described with reference to components of system (100) shown in FIGS. 1 to 3.

Step (402) comprises using first sensors to generate a plurality of time-separated training values of the applicable first AP, and using a second sensor to generate a plurality of time-separated training values of the second AP. “Time-separated”, as used herein means that the plurality of values are measured at different times. Each of the measured training values is uniquely time-paired with a different one of the training values of the first APs. “Time-paired” as used herein means that the values of the first AP and the second APs are measured at the same time or substantially the same time, such that the generated first and second AP values can be considered as representing a simultaneous state of the first AP and the second AP in an air sample, in the vicinity of physically attached first and second sensors. Each of the training values is “uniquely” time-paired in the sense that each training value of the second AP is time-paired to only one value of the first AP, and that the values of the second AP are time-paired to different values of the first AP. In other words, there is a one-to-one mapping of the training values of the first AP to the training values of the second AP.

In one embodiment, step (402) is implemented by the processor (202) executing instructions on memory (204) of sensor unit (200) to control NO₂, O₃, T and CO sensors (208 to 222) to measure training values of the applicable first and second APs, at fixed time intervals, as governed by a computer clock. The measured training values of the first and second APs may be time-paired by being stored in a data structure linking the first and second APs, or by being stored in association with a common date/time stamp, or other temporal index, which may or may not explicitly indicate the time that training values were measured. It will be appreciated that the time-pairing may be performed after the training values of the first and second APs have been generated.

Optionally, step (402) may comprise using an accuracy factor sensor to generate time-separated values of an accuracy factor such as a speed, v₍ₖ₎, of the sensor unit (200), that affects the accuracy of the first and/or second APs, and that are uniquely time-paired with the training values of the first and second APs.

The time-paired measured training values of the first and second APs, and optionally the accuracy factor(s), are shown by the notation (CO, NO₂, O₃, T, v)(k), under the heading “Training Mode Output” in FIG. 2 showing the sensor unit, and under the heading “Training Mode Input” in FIG. 3 showing the server computer. (The term “Training Mode” is used for convenient discussion to refer to the operation of the sensor unit and the server computer during this training process, in distinction to the operation of the sensor unit and server computer during a “Test Mode” as described below in respect to steps (408)-(414)). The subscript (k) is an index denoting the temporal index of the time-paired measured training values. The processor (202) controls data communication module (224) of sensor unit (200) to transmit the time-paired training values, and optionally the accuracy factor values, to cloud database (14) where they are stored.

Optional step (404) comprises using a computer system to pre-process the training values of first and second APs. In one embodiment, step (404) is implemented by the processor (302) executing instructions stored on memory (304) of server computer (300) to control data communications module (310) to query the cloud database (14) to access the stored training values of the time-paired first and second APs. The processor (302) executes an algorithm of the training data pre-processing module (312) stored in the memory (304) to perform operations on the training values so that they can be used for training the air quality estimation/prediction model (310). In some embodiments, the pre-processing operations may comprise deriving additional values from the training data for use with a particular air quality estimation/prediction model. For example, as illustrated by Example no. 1 described below, optional step (402) may comprise using the accuracy factors to calculate trust coefficients, R₍ₖ₎, that are associated with the time-paired training values of the first and second APs. In other embodiments, the pre-processing operations may comprise formatting the training data so that it is suitable for use with a particular air quality estimation/prediction model, as illustrated by the vector of time-separated second APs in Example no. 2 described below.

Step (406) comprises using the computer system to train an air quality estimation/prediction model for estimating or predicting an output value of the first APs based on at least one measured input value of the second AP, wherein the training is based on the training values of the first and second APs, optionally weighted by the trust coefficients, R₍ₖ₎. This training step is performed to increase the accuracy of the value of a first AP estimated or predicted by the air quality estimation/prediction model (310). The present invention is not limited by any particular required accuracy level of the trained air quality estimation/prediction model, which may depend on factors such as the target response, and the number of training values of the first and second AP used for training of the air quality estimation/prediction model. The present invention is also not limited by the number of training values of the first and second AP used for training of the air quality estimation/prediction model, which may depend on factors such as the type of first and second APs, and a particular sample of the training values. As an example, tens of thousands of training values may be used for training of the air quality estimation/prediction model. In one embodiment, step (406) is implemented by the processor implementing an algorithm of training module (314) to operate on air quality estimation/prediction model (310) stored in memory (304) of server computer (300). In embodiments, the air quality estimation/prediction model (310) may be a machine learning model, and the training algorithm may implement machine learning techniques, known to persons skilled in the art of artificial intelligence. As non-limiting examples, the machine learning model may have an architecture in the form of an artificial neural network (ANN), a regression model including a linear and/or non-linear regression model, decision-tree model, support-vector machine classification models, or Bayesian or belief network model, among others. Training algorithms are known to persons skilled in the art of machine learning. The selection of a training algorithm that is suitable for a particular machine learning model is within the skill of persons of ordinary skill in the art. In embodiments, the machine learning model and training algorithms may be implemented using available software environments or platforms, with non-limiting examples including Google Colaboratory™ (also known as Colab™) (Google Research), Juptyer™ (also known as IPython™) (Project Jupyter), and Anaconda™ (Anaconda, Inc.; Austin, Tex., USA).

In one example, the machine learning model is an ANN, and the accuracy of the estimated or predicted value of the first AP is quantified by a loss function that measures how the output of the ANN matches the desired output of the measured output values of the first AP. The training algorithm adjusts parameters of the air quality estimation/prediction model to minimize the value of the loss function, which is when the air quality estimation/prediction model is considered to be “trained.” Among the parameters that are adjusted are the number of “layers” that make up the ANN, and the number of “neurons” or “nodes” within each layer. These parameters may be pre-assigned by an operator prior to training the ANN, and then adjusted through an iterative process.

Step (408) comprises using the second sensor to generate at least one measured input value of the second AP. In one embodiment, step (408) is implemented by the processor (202) executing instructions on memory (204) of sensor unit (200) to control CO concentration sensor (208) to measure the input value of CO concentration. The measured input value(s) of the second APs is shown by the notation CO₍ₙ₎, under the heading “Test Mode Output” in FIG. 2 showing the sensor unit, and under the heading “Test Mode Input” in FIG. 3 showing the server computer. The subscript (n) denotes a temporal index of the measured input value of the second AP. In some embodiments, the measured input value of the second AP consists of a single measurement, as illustrated by Example no. 1 described below. In other embodiments, the measured input values of the second AP comprises a vector of a plurality of time-separated measurements, as illustrated by Example no. 2 described below, and shown by the vector notation \vec{CO}ₗ(i) for i=1 to n under the heading “Test Mode Input” in FIG. 3. The processor (202) controls data communication module (224) of sensor unit (200) to transmit the input value of the second AP to cloud database (14) where it is stored.

Optional step (410) comprises using the computer system to pre-process the at least one input input value of the second AP. In one embodiment, step (410) is implemented by processor (302) executing instructions stored on memory (304) of server computer (300) to control data communications module (310) to query the cloud database (14) to access the stored measured input value(s) of the second AP, CO₍ₙ₎. The processor (302) executes an algorithm of input data pre-processing module (316) stored in memory (304) to perform operations on the input value of the second AP so that it can be used as input for the air quality estimation/prediction model (310). In some embodiments, the pre-processing operations may comprise formatting the input values of the second AP so that they are suitable for use with a particular air quality estimation/prediction model, as illustrated by Example no. 2 described below.

Step (412) comprises using the computer system to estimate or predict the output value of the first AP by applying the trained air quality estimation/prediction model to the at least one measured input value of the second AP. In one embodiment, step (412) is implemented by the processor (302) executing instructions of estimation/prediction module (318) stored in memory (304) to provide the at least one measured input value of the second AP as an input to the air quality estimation/prediction model (310), which is used to compute an output comprising an estimated or predicted output value of the first APs. In some embodiments, the estimated or predicted output value of the first AP is an estimated output value for the same temporal index of the measured input value of the second AP, as illustrated in Example no. 1 described below; this is shown by the notation (NO₂, O₃, T)₍ₙ₎ under the heading “Test Mode Output” in FIG. 3. In other embodiments, the estimated or predicted output value of the first AP is a predicted output value for a temporal index that is subsequent to the temporal index or indices of the measured input value(s) of the second AP, as illustrated in Example no. 2 described below; this is shown by the notation (NO₂, O₃, T)₍ₙ₊₁₎ under the heading “Test Mode Output” in FIG. 3.

Step (414) comprises using the computer system to transmit the estimated or predicted output value of the first AP to another computer, machine, or device, and/or display the estimated or predicted value of the first AP on a display device. In one embodiment, step (412) is implemented by processor (302) executing instructions stored on memory (304) of server computer (300) to control data communications module (308) to transmit the estimated or predicted output value of the first AP to the cloud database (14) where it can be accessed by other computer systems. Display device (310) of the server computer (300) or of another computer system accessing the cloud database (14) may display the estimated or predicted output value of the first AP, in a human-readable form such as a textual or a graphical representation.

In embodiments of the method, steps (408) to (414) are performed in real-time. That is, the elapsed time from the measurement of the input value of the second AP in step (408) to the output of step (414) is practically instantaneous, being practically limited only by any latency in the communication and processing of the sensor data and output.

### Alternative and Specific Embodiments

In the embodiment of the method (400) described above, the first APs are each of NO₂ concentration, O₃ concentration, and air temperature. In other embodiments, the first AP may be one of these APs, or any combination of them. In still other embodiments, the first AP may be a concentration of another gaseous component of air, or a measure of air quality such as airborne particles.

In the embodiment of the method (400) described above, the second AP is CO concentration. In other embodiments, the second AP may be a concentration of another gaseous component of air or a measure of air quality such as airborne particles.

In the embodiments of the method (400) described above, steps (404), (406), (410), (412), and (414) are implemented by processor (302) executing instructions stored on memory (304) of server computer (300). In other embodiments, any one or more of these steps may be implemented by processor (202) executing instructions stored on memory (204) local to sensor unit (200). In such embodiments, the processing of these steps may be performed “offline” (without a need for a communications network to transmit the data to another computer system), and the results of step (412) may be stored in the memory (204) or another memory local the sensor unit (200) for retrieval or downloading at a later time. In still other embodiments, the instructions executed by either or both of the processors (202, 302) may be stored entirely either on memory (204) or memory (304), or in a combination of memories (204, 304). Accordingly, in embodiments, any one or more of these steps, or further computational steps described in additional embodiments and examples below, may be implemented entirely by processor (302) of server computer (300), entirely by processor (202) of sensor unit (200), or by a combination of the processors (202, 302) executing instructions stored entirely either on memory (204) of sensor unit (200) or on memory (304) of server computer (300), or on a combination of memories (204, 304). Any combination of processor(s) and memory or memories implementing these steps may be considered as the “computer system” in the method shown in FIG. 4, and other methods described herein.

In the embodiment of the method (400) described above, step (408) of generating the at least one measured input value of the second AP is performed using the same second sensor used in step (402) to generate the training values of the second AP. In other embodiments, step (408) of generating the at least one input value of the second AP may be performed using a different sensor, referred to herein as a “third sensor”. For example, referring to FIG. 1, the second sensor used in step (402) may be a CO concentration sensor of one sensor unit (200a) that either stationary, or a mobile sensor, or mounted to one mobile platform, such as vehicle (10a), while the third sensor used in step (408) may be a CO concentration sensor of a different sensor unit (200b), which may be stationary, or a mobile sensor, or mounted to a different mobile platform, such as another vehicle (10b). If the second and third sensors are alike in construction, then the training model trained with the training values generated by the second sensor in step (406) can be applied to measured input values of the second AP generated by the third sensor. Accordingly, sensor unit (200a) may be used as a “benchmark” sensor unit for generating training values of the first and second APs, and optionally measured input values of the second AP. In embodiments, the generation of training values may be performed by a manufacturer or supplier of the sensor units (200) (e.g., as part of a manufacturing process, or as part of the supply of a software-as-a-service (SAAS) delivery model), or by an end user (e.g., a municipality or environment monitoring agency) of the sensor units (200). Sensor unit (200b) may be used as a “test” sensor unit used for generating input values of the second AP during the “Test Mode”. Accordingly, sensor unit (200b) may omit sensor(s) for generating the first APs—i.e., NO₂ concentration sensor (208), O₃ concentration sensor (210) and air temperature sensor (212) in the embodiment shown in FIG. 2.

In the embodiment of the method (400) described above, step (408) comprises generating a measured input value of the second AP. In embodiments, step (408) may also comprise using the first sensor to generate a measured test value of the first AP, denoted in FIG. 4 as (NO₂, O₃, T)_{(n′)}). As shown by return loop (416), the measured test value of the first AP and the measured input value of the second AP may be added to the training values of the first and second APs to augment the training values for training the air quality estimation/prediction model (310) in a subsequent performance of step (406). Accordingly, it will be understood that steps (402) through (406) may be performed repeatedly on an augmented set of training values or a new set of training values, so that training of the air quality estimation/prediction model (310) is continuously performed after measured output values of the first AP and input values of the second AP (s) are obtained when step (408) is performed.

### EXAMPLES

The following three examples illustrate specific aspects of exemplary embodiments of the method (400) described above.

### Example No. 1: Calibration

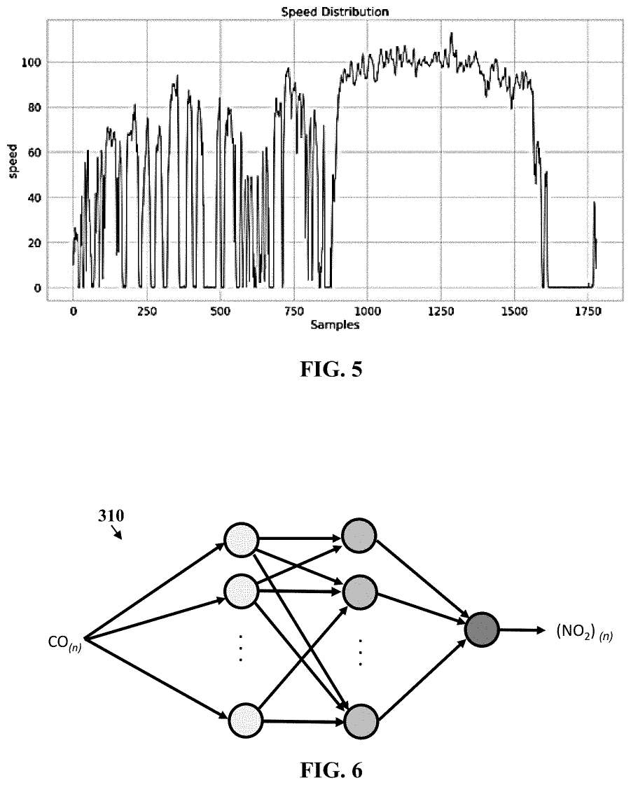

FIG. 5 is a chart showing an example of speed variation for a sensor unit (200) mounted on a mobile platform in the form of a vehicle over a plurality of time-separated air quality samples collected over a vehicle trip on a typical highway drive. The accuracy of the NO₂ concentration values measured by the NO₂ concentration sensor (208) is expected to vary as accuracy factor(s) such as the speed, wind speed, wind direction, temperature, ambient air pressure, and/or relatively humidity, encountered by the sensor unit (200) vary over the vehicle trip. For example, it is expected that the accuracy of the measured NO₂ value degrades with the increase in the speed of the sensor unit (200). In contrast, the accuracy of CO concentration values (not shown in FIG. 5) measured by the CO concentration sensor (214) tends to be more stable even with speed variations.

In one embodiment, this phenomenon is exploited to calibrate the measured NO₂ concentration values. In this embodiment, the first AP is NO₂ concentration, the second AP is CO concentration, and the accuracy factor is speed of the sensor, v₍ₖ₎. It will be understood that this embodiment may be adapted for different combinations of first and/or second APs, and the accuracy factor(s).

At step (402), the time-paired training values of the first and second APs is in the form (NO₂, CO)₍ₖ₎. At step (404), the computer system pre-processes the training values (NO₂)₍ₖ₎ to derive a corresponding trust coefficient (R)₍ₖ₎ that reflects how accurate the training value, (NO₂)₍ₖ₎, is expected to be—i.e., how “trustable” it is. A higher trust coefficient (R)₍ₖ₎ indicates that the training value (NO₂)₍ₖ₎ is more trustable, and means that the particular training value sample (NO₂, CO)₍ₖ₎ is assigned a greater weight than other training value samples having lower trust coefficients, when training the air quality estimation/prediction model (310) in step (406). Step (404) results in training values in the form (NO₂, CO, R)₍ₖ₎ for use as input in training step (406).

In one non-limiting embodiment, the determination of the trust coefficient, R₍ₖ₎, is based on the speed, v₍ₖ₎, of the sensor unit (200) when the training values of the first and second APs are measured by the first and second sensors. To explain, the measurements generated by sensors (208 to 222) of mobile sensor unit (200) are expected to more closely match the measurements generated by a stationary sensor unit (200) when the environment of the mobile sensor unit (200) is more stable. In one embodiment, the stability of the environment can be defined, at least in part, by the speed of the mobile sensor unit (200)—that is, as the speed of the mobile sensor unit (200) increases, the environment is considered to be less stable. In one embodiment, the processor (204) of the sensor unit (200) receives data from the GPS module (226) and/or the mobile platform data interface module (228) that either indicates the speed of the sensor unit (200), or permits the speed of the sensor unit (200) to be computed at the time the training values of the first and second APs were measured by the first and second sensors. In this manner, the speed, v₍ₖ₎, of the sensor unit (200) is also time-paired with the training values of the first and second APs. For example, in one embodiment, the GPS module (226) may generate location coordinates for the sensor unit (200) that are time-paired with measured training values of the first and second APs. The computer system (e.g., processor (202) and/or (302)) and/or the GPS module (226) uses the location information to compute speed of the sensor unit (200) using known kinematic relationships (e.g., speed=distance/time). As another example, in another embodiment, the mobile platform data input interface (228) may interface with a telematics system of a mobile platform (e.g., a vehicle) to directly receive data indicative of the speed of the mobile platform which is used as the speed of the sensor unit (200); again, this data is time-paired with the measured training values of the first and second APs. The aforementioned GPS module (226) and telematics system of the mobile platform are exemplary embodiments of “accuracy factor sensors”. Other non-limiting embodiments of “accuracy factor sensors” may include thermometers where the accuracy factor(s) comprise air temperature, an anemometer where the accuracy factor(s) comprise wind speed and/or direction, or a hygrometer where the accuracy factor(s) comprise relatively humidity.

The computer system computes the trust coefficient, R₍ₖ₎, based on a rule stored in a memory (e.g., expressed by mathematical formulae, and/or logical relationships) relating the trust coefficient, R₍ₖ₎, and the speed, v₍ₖ₎, of the sensor unit (200). The present invention is not limited by any particular rule. In embodiments, the rule may be based on empirical testing of sensor units (200), and/or theoretical relationships. As a non-limiting example, the rule may define an inverse relationship with between the trust coefficient, R₍ₖ₎, and the speed, v₍ₖ₎, of the sensor unit (200), such as of the form R₍ₖ₎∝1/(v₍ₖ₎), so that the value of the trust coefficient decreases as the speed of the sensor unit (200) increases. Therefore, a higher trust coefficient will be assigned to training data generated when the sensor unit (200) is stationary (e.g., when mounted to a vehicle stopped at a traffic light) or moving at a relatively low speed (e.g., when the sensor unit (200) is part of a portable device moved by a walking pedestrian), as compared with training data generated when the sensor unit (200) is moving at high speed (e.g., when mounted to a vehicle travelling on a highway).

As shown schematically in FIG. 6, in some embodiments, the air quality estimation/prediction model (310) is in the form an artificial neural network (ANN) which has three dense layers. As used herein, a “dense layer” refers to a component of the model that models a non-linear mapping between an input and output for the layer. The input is a single measured input value of CO concentration, denoted as (CO)₍ₙ₎. The output is a single estimated or predicted output value of NO₂ concentration, denoted as (NO₂)₍ₙ₎. The trust coefficients, R₍ₖ₎, are applied as weightings to the air estimation/prediction model (310) during the training step (406), but preferably the trust coefficients are not used as an input during the estimation/prediction step (412).

As a result of the air estimation/prediction model (310) having been trained on training data that is weighted by trust coefficients, (R)₍ₖ₎, the estimated output value of (NO₂)₍ₙ₎ concentration may have a higher accuracy than would result from measurement of the NO₂ concentration using the NO₂ concentration sensor (208), particularly when that sensor is travelling at higher speeds. Accordingly, the estimated output value, (NO₂)₍ₙ₎, may be used to calibrate or evaluate the accuracy of a test value of NO₂ concentration measured by sensor (208) at the same time as when sensor (214) is used to measure the input value of CO concentration.

### Example No. 2: Forecasting

This example illustrates an embodiment of the present invention that predicts a future output value of a first AP for forecasting purposes, based on historical measured input values of a second AP. In this example, each of NO₂ concentration, O₃ concentration, and air temperature is a first AP, and CO concentration is the second AP. It will be understood that this embodiment may be adapted for different first and/or second APs.

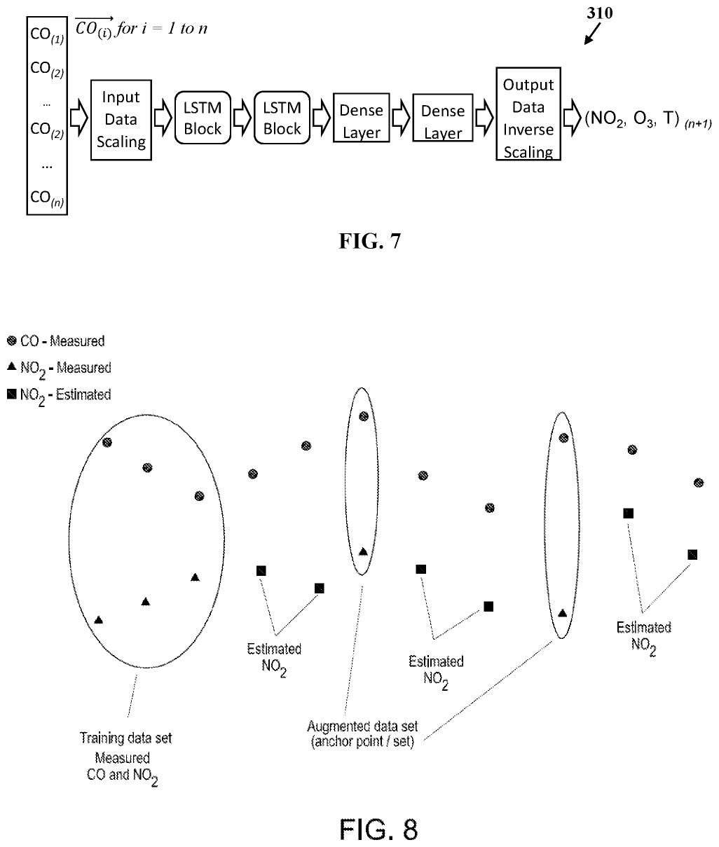

FIG. 7 shows schematically the air quality estimation/prediction model (310) used in this embodiment of the method. The input is a series of time-separated input values of CO concentrations, denoted by the vector \vec{CO}ₜ(i) for i=1 to n. The outputs are a single predicted output value of NO₂ concentration, denoted NO₂₍ₙ₊₁₎, a single predicted output value of O₃ concentration, denoted O₃₍ₙ₊₁₎, and a single value of air temperature denoted T₍ₙ₊₁₎. In one non-limiting embodiment, the value of n is 60. That is, the air quality estimation/prediction model (30) forecasts the output values of NO₂ concentration, O₃ concentration, and air temperature at a future 61^{st} temporal index, based on measured input values of CO concentration at the historical preceding 60 temporal indices. Accordingly, at steps (404) and (410), the computer system pre-processes the training values (CO)₍ₖ₎ and input values (CO)₍ₙ₎ to format them into the vector \vec{CO}ₗ(i) for i=1 to 60.

In the embodiment shown in FIG. 7, the air quality estimation/prediction model (310) is in the form an artificial neural network (ANN) comprising an input data scaling block, two long short-term memory (LSTM) blocks, two dense layers, and an output data inverse scaling block. As known in the art, LSTM blocks enable processing sequential data (i.e., the vector \vec{CO}ₗ(i) of n time-separated values) using an input, output and forget gate. The input scaling block may apply a scaling factor or normalize the input data before it is processed by the subsequent blocks and layers, to increase the accuracy of the air quality estimation/prediction model (310) and/or to increase the speed and/or the stability of the training of the air quality estimation/prediction model (310). The output data inverse scaling block applies the inverse of the scaling factor to the output of the preceding blocks and layers to reverse the effect of the input scaling block. Data scaling algorithms suitable for use in machine learning are known in the art.

### Example no. 3: Augmentation of Training Data

This example, as shown in FIG. 8, illustrates an embodiment of the present invention where the original data that is used to train the air quality estimation/prediction model (310) gets augmented with a small data set, in order to improve accuracy or to extend the range of the prediction. These future small data sets may comprise additional measured values, or may comprise artificial or synthetic values generated programmatically, and can provide anchor points for the training model to provide better accuracy or prevent the model from diverging over an extended estimation/prediction range.

### Interpretation

Aspects of the present invention may be described with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

The flowchart and block diagrams in the Figures illustrate the architecture, functionality, and operation of possible implementations of systems, methods and computer program products according to various embodiments of the present invention. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The corresponding structures, materials, acts, and equivalents of all means or steps plus function elements in the claims appended to this specification are intended to include any structure, material, or act for performing the function in combination with other claimed elements as specifically claimed.

References in the specification to “one embodiment”, “an embodiment”, etc., indicate that the embodiment described may include a particular aspect, feature, structure, or characteristic, but not every embodiment necessarily includes that aspect, feature, structure, or characteristic. Moreover, such phrases may, but do not necessarily, refer to the same embodiment referred to in other portions of the specification. Further, when a particular aspect, feature, structure, or characteristic is described in connection with an embodiment, it is within the knowledge of one skilled in the art to affect or connect such module, aspect, feature, structure, or characteristic with other embodiments, whether or not explicitly described. In other words, any module, element or feature may be combined with any other element or feature in different embodiments, unless there is an obvious or inherent incompatibility, or it is specifically excluded.

It is further noted that the claims may be drafted to exclude any optional element. As such, this statement is intended to serve as antecedent basis for the use of exclusive terminology, such as “solely,” “only,” and the like, in connection with the recitation of claim elements or use of a “negative” limitation. The terms “preferably,” “preferred,” “prefer,” “optionally,” “may,” and similar terms are used to indicate that an item, condition or step being referred to is an optional (not required) feature of the invention.

The singular forms “a,” “an,” and “the” include the plural reference unless the context clearly dictates otherwise. The term “and/or” means any one of the items, any combination of the items, or all of the items with which this term is associated. The phrase “one or more” is readily understood by one of skill in the art, particularly when read in context of its usage.

The term “about” can refer to a variation of ±5%, ±10%, ±20%, or ±25% of the value specified. For example, “about 50” percent can in some embodiments carry a variation from 45 to 55 percent. For integer ranges, the term “about” can include one or two integers greater than and/or less than a recited integer at each end of the range. Unless indicated otherwise herein, the term “about” is intended to include values and ranges proximate to the recited range that are equivalent in terms of the functionality of the composition, or the embodiment.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc.

As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio.

The claimed invention is:

1. A method for estimating or predicting an output value of a first ambient parameter (AP) based on at least one measured input value of a second AP, wherein the second AP is different from the first AP, the method comprising the steps of:
(a) generating a plurality of time-separated training values of the first AP, a plurality of time-separated training values of the second AP, and a plurality of time-separated values of an accuracy factor that affects an accuracy of measured values of either one or both of the first and second APs, wherein each of the training values of the second AP and the accuracy factor are time-paired uniquely with one of the training values of the first AP;
(b) using a computer system, for each of the time-paired training values of the first and second APs, calculating an associated trust coefficient based on the value of the accuracy factor that is uniquely time-paired to the training values of the first and second APs;
(c) using the computer system, training an air quality estimation/prediction model for estimating or predicting the output value of the first AP based on the at least one measured input value of the second AP, wherein the training is based on the training values of the first and second APs weighted by the associated trust coefficients;
(d) measuring the at least one measured input value of the second AP; and
(e) using the computer system, estimating or predicting the output value of the first AP by applying the trained air quality estimation/prediction model to the at least one measured input value of the second AP.

2. The method of claim 1 wherein the accuracy factor comprises a speed of a sensor for generating either the first AP training values or the second AP training values.

3. The method of claim 2 wherein the speed of the sensor is based on location information generated by a GPS module.

4. The method of claim 2 wherein the speed of the sensor is acquired from a telematics system of a mobile platform carrying the sensor.

5. The method of claim 1 wherein the accuracy factor comprises wind speed, air temperature, ambient air pressure, or relatively humidity.

6. The method of claim 1 wherein the accuracy factor comprises a combination of any two or more of: a speed of a sensor for generating either the first AP training values or the second AP training values; wind speed; air temperature; ambient air pressure; and relative humidity.

7. The method of claim 1, wherein generating the first AP training values comprises measuring the first AP training values using a first sensor mounted on a mobile platform; and generating the second AP training values comprises measuring the second AP training values using a second sensor mounted on the mobile platform.

8. The method of claim 7, wherein measuring at least one measured second AP input value is performed using the second sensor.

9. The method of claim 7, wherein measuring at least one measured second AP input value is performed using a third sensor.

10. The method of claim 1 wherein step (e) comprises estimating the output value of the first AP for a time corresponding to when at least one measured input value of the second AP is measured in step (d).

11. The method of claim 10 comprising the further step of using the estimated output value of the first AP to calibrate the accuracy of a measured test value of the first AP.

12. The method of claim 1 wherein step (e) comprises predicting the output value of the first AP for a time subsequent to when the at least one measured input value of the second AP is measured in step (d).

13. The method of claim 1, wherein the first AP comprises one or more of NO₂ concentration, O₃ concentration, or air temperature.

14. The method of claim 1 wherein the second AP comprises CO concentration.

15. The method of claim 1 wherein the air quality prediction model comprises a machine learning module.

16. The method of claim 15 wherein the machine learning module comprises an artificial neural network.

17. The method of claim 16 wherein the artificial neural network comprises at least one dense layer.

18. The method of claim 16 wherein the artificial neural network further comprises an input data scaling block, at least one long short-term memory block, and an output data inverse scaling block.

19. The method of claim 1 comprising the further step of periodically augmenting the air quality estimation/prediction model with a measured or artificial data set.

20. A system for estimating or predicting an output value of a first ambient parameter (AP), based on at least one measured input value of a second AP, wherein the second AP is different from the first AP, the system comprising:
a first sensor for measuring values of the first AP;
a second sensor for measuring values of a second AP, wherein the first and second sensors are physically proximate to each other;
an accuracy factor sensor for measuring values of an accuracy factor that affects an accuracy of measured values of either one or both of the first and second APs;
a computer system comprising: a processor operatively connected to the first sensor to access the values of the first AP, to the second sensor to access values of the second AP, and to the accuracy factor sensor to access values of the accuracy factor; and a non-transitory computer readable medium storing instructions that, when executed by the processor, cause the processor to perform a method comprising the steps of:
(a) accessing a plurality of time-separated training values of the first AP, the second AP, and the accuracy factor, wherein each of the training values of the second AP and the accuracy factor are time-paired uniquely with a one of the training values of the first AP;
(b) for each of the time-paired training values of the first and second APs, calculating an associated trust coefficient based on the value of the accuracy factor that is uniquely time-paired with the training values of the first and second APs;
(c) training an air quality estimation/prediction model for estimating or predicting the output value of the first AP based the on at least one measured input value of the second AP, wherein the training is based on the time-paired training values of the first and second APs weighted by the associated trust coefficients;
(d) accessing the at least one measured input value of the second AP; and
(e) estimating or predicting the output value of the first AP by applying the trained air quality estimation/prediction model to the at least one measured input value of the second AP.

21. The system of claim 20 wherein the accuracy factor comprises a speed of the first sensor or the second sensor, or both.

22. The system of claim 21 wherein the accuracy factor sensor comprises a GPS module.

23. The system of claim 21 wherein the accuracy factor sensor comprises a telematics system of a mobile platform carrying the first sensor or the second sensor, or both.

24. The system of claim 20 wherein the accuracy factor comprises wind speed, air temperature, relatively humidity, or ambient air pressure.

25. The system of claim 20 wherein the accuracy factor comprises a combination of any two or more of: a speed of a sensor for generating either the first AP training values or the second AP training values; wind speed; air temperature; ambient air pressure; and relative humidity.

26. The system of claim 20 wherein the first and second sensors are mounted on a mobile platform.

27. The system of claim 20 wherein step (e) comprises estimating the output value of the first AP for a time corresponding to when the at least one measured input value of the second AP is measured.

28. The system of claim 27 wherein the method further comprises the step of using the estimated output value of the first AP to calibrate the accuracy of a measured test value of the first AP.

29. The system of claim 20 wherein step (e) comprises predicting the output value of the first AP for a time subsequent to when at least one measured input value of the second AP is measured.

30. The system of claim 20 wherein the first AP comprises one or more of NO₂ concentration, O₃ concentration, or air temperature.

31. The system of claim 20 wherein the second AP comprises CO concentration.

32. The system of claim 20 wherein the air quality estimation/prediction model comprises a machine learning model.

33. The system of claim 32 wherein the machine learning model comprises an artificial neural network.

34. The system of claim 33 wherein the artificial neural network comprises at least one dense layer.

35. The system of claim 33 wherein the artificial neural network further comprises an input data scaling block, at least one long short-term memory block, and an output data inverse scaling block.

36. The system of claim 20 wherein the air quality estimation/prediction model is periodically augmented with a measured or artificial data set.
